(19) United States
(12) Patent Application Publication
(10) Pub. No.: US 2001/0051628 A1
(43) Pub. Date: Dec. 13, 2001

(54) METHODS TO MODULATE THE RESISTANCE OF CELLS TO APOPTOSIS MEDIATED BY MUTANT EPIDERMAL GROWTH FACTOR RECEPTORS

(76) Inventors: H.-J. SU HUANG, RANCHO SANTA FE, CA (US); MOTOO NAGANE, SAN DIEGO, CA (US); WEBSTER K. CAVENEE, LA JOLLA, CA (US); ALEXANDER LEVITZKI, BURLA (IL); AVIV GAZIT, NOF HARIM (IL)

Correspondence Address: MORGAN LEWIS & BOCKIUS, 1800 M STREET NW, WASHINGTON, DC, 200365869

(21) Appl. No.: 09/071,541

(22) Filed: May 4, 1998

(51) Int. Cl. A61K 31/33; A61K 31/505; A61K 33/24; C07D 239/00; C07D 491/00; A61K 31/44

(52) U.S. Cl. 514/259; 514/183; 514/449; 424/649; 423/351; 540/478; 544/253

(57) ABSTRACT

The present invention provides methods and compositions for enhancing the activity of various therapeutic agents that induce apoptosis by modulating the apoptosis-inhibiting effects of the expression products of mutant epidermal growth factor receptor (EGFR) genes. Methods and compositions of particular use in the treatment of cancers, such as glioma, that express such a mutant EGFR gene are provided.

### TECHNICAL FIELD

The present invention generally relates to methods for suppressing the apoptosis-inhibiting activity of the expression products of mutant epidermal growth factor receptor (EGFR) genes. It specifically relates to methods and compositions useful in the treatment of cancers, such as gliomas, that typically express such mutant EGFR genes. Tyrosine kinase inhibitors, preferably those that are selective for tumor specific mutant EGFRs, particularly ΔEGFR, are administered in combination with other therapies that induce apoptosis in such tumor cells.

### BACKGROUND OF THE INVENTION

A. Programmed Cell Death

Programmed cell death (sometimes referred to as apoptosis) is distinguishable, both morphologically and functionally, from necrosis. Programmed cell death is a natural form of death that organisms use to dispose of cells. Cells dying by programmed cell death usually shrink, rarely lyse, and are efficiently removed from the organism without the appearance of inflammation. Necrosis, however, is a pathological type of cell death observed following physical or chemical injury, exposure to toxins or ischemia (lack of oxygen). Cells undergoing such programmed cell death are rapidly recognized and engulfed by macrophages (Michael Hengartner, “Cell Death and Aging, Molecular Mechanisms of,” IN MOLECULAR BIOLOGY AND BIOTECHNOLOGY 158-62 (ed. R. A. Meyers, 1995)).

Apoptosis was initially used to describe a subset of programmed cell deaths sharing a particular set of morphological features which include membrane blebbing, shrinkage of cytoplasm, chromatic condensation and formation of a “DNA ladder.”

During apoptosis, cells lose their cell junctions and microvilli, the cytoplasm condenses and nuclear chromatin marginates into a number of discrete masses. While the nucleus fragments, the cytoplasm contracts and mitochondria and ribosomes become densely compacted. After dilation of the endoplasmic reticulum and its fusion with the plasma membrane, the cell breaks up into several membrane bound vesicles, referred to as apoptotic bodies, which are usually phagocytosed by adjacent cells. Activation of particular genes such as tumor suppressor genes in vertebrates, is thought to be necessary for apoptosis to occur. Apoptosis induced by numerous cytotoxic agents can be suppressed by expression of the gene bcl-2, which produces a cytoplasmic protein Bcl-2 (THE ENCYCLOPEDIA OF MOLECULAR BIOLOGY 67, ed. John Kendrew et al., Blackwell Science; Oxford, England, 1994).

B. Gliomas and Drug Resistance

Malignant glioma, the most common primary brain tumor, remains clinically intractable despite intensive treatment regimens. Persistent invasion of these tumor cells into the adjacent normal brain parenchyma renders surgical resection incomplete, and necessitates adjuvant treatments such as radiation and chemotherapy (1). Most gliomas, however, eventually become drug-resistant limiting the effectiveness of brain tumor chemotherapy.

There are a number of mechanisms that may contribute to cellular drug resistance, including reduced intracellular drug concentrations, rapid inactivation of the drug, and increased rate of DNA repair (2). Inhibition of apoptosis, a genetically controlled form of cell death, may also be important for drug resistance because the primary mechanism by which most chemotherapeutic agents having disparate modes of action and cellular targets induce cell death appears to be apoptosis (3). The observations that tumors which were either deficient in p53, a tumor suppressor that plays an important role in the induction of apoptosis, or those in which expression of the anti-apoptotic protein Bcl-2 was elevated, were resistant to apoptosis and showed poor response to radiotherapy and chemotherapy (4, 5) suggest that tumor-specific genetic lesions may bestow this property to tumor cells, resulting in a survival advantage.

It has become clear that the malignant progression of gliomas involves accumulation of genetic alterations that inactivate tumor suppressor genes such as p53, p16, RB, and PTEN, or activate oncogenes including the epidermal growth factor receptor (EGFR), CDK4, and MDM2 genes, both of which lead to a growth advantage to tumor cells (6, 7). EGFR gene amplification occurs frequently in gliomas and is restricted to high grade tumors, occurring at a frequency of 40-60% of grade IV gliomas (glioblastoma) (8, 9). Several clinical and histopathological studies have shown that the presence of EGFR amplification correlates with a shorter interval to relapse of the disease and lower rates of survival in patients receiving adjuvant therapies, suggesting that they may affect responsiveness of malignant gliomas to treatment (9).

The majority of the tumors that harbor EGFR gene amplifications also show rearrangements of the gene (8, 10) most commonly genomic deletion of exons 2-7, resulting in a mutant receptor having a truncation in its extracellular domain (ΔEGFR or EGFRvIII) (10, 11). This specific genetic alteration has also been found frequently in lung and breast cancers (12, 13). Our previous studies showed that introduction of ΔEGFR into the U87MG human glioma cell line, U87MG.ΔEGFR, resulted in cell surface expression of a truncated receptor having a ligand-independent, weakly constitutively active and unattenuated kinase, and enhanced tumorigenicity in nude mice (14) which was mediated by both an increase in proliferation and a decrease in apoptosis of tumor cells.

In contrast, overexpression of wild-type (wt) EGFR was unable to confer a similar growth advantage (15, 16). Bcl-X_{L}, an inhibitor of the Bcl-2 family of apoptotic proteins, was up-regulated in U87MG.ΔEGFR tumors, which was inversely correlated with their reduced apoptotic rate (16). Overexpression of Bcl-X_{L} has been shown to confer a drug resistant phenotype in some tumor cells (17, 18) and also to suppress activation of caspases, the cysteine proteases that specifically cleave proteins after Asp residues and are thought to play a key role in the execution phase of apoptosis (19).

C. Gliomas and Anti-Cancer Therapies

A variety of chemotherapeutic agents are utilized for the treatment of glioma. However, as described for example in a 1995 review article by Chang et al. (39), the prognosis of patients with malignant gliomas remains poor, notwithstanding recent advances in surgical and radiation therapy techniques. Few agents have been developed specifically for brain tumors, and the nitrosoureas remain the most active antitumor agents available.

Resistance to chemotherapy or radiotherapy is a major obstacle for the treatment of malignant gliomas that are surgically incurable because of their diffusely infiltrative nature. In addition to the conventional drug-resistance mechanisms related to pharmacodynamics, suppression of apoptosis is another means of acquiring drug resistance, and could be mediated by specific genetic alterations in tumor cells, such as inactivating mutations of p53 and/or overexpression of Bcl-2 (3-5).

Because chemotherapeutic regimens in present use for glioma have substantial toxicity, example, experimental research also has been directed to identifying other therapeutic agents. Thus, in one study, the maximum tolerable dose (MTD) of thiotepa (TT) that can be administered with etoposide without stem cell support was evaluated. Chang et al. (37) studied the efficacy and toxicity of the combination of high-dose tamoxifen and interferon alpha in adults with recurrent glioma in a phase II trial. Both agents reportedly had more acceptable toxicity profiles and can be administered in an outpatient setting.

Experimental research also has been directed to increasing the efficacy of existing chemotherapeutic agents. For example, cisplatin (CDDP), which has toxic side effects, is commonly used for the treatment of glioma. Thus, a recent study showed that, in CDDP-treated animals, pre-treatment with LTC4 increased survival to a statistically significant degree (42). The administration of EGF also was found to significantly enhance cisplatin-induced tumor cell kill as reflected by a further decrease in the final volume of tumors following treatment with EGF plus cisplatin relative to tumors treated with cisplatin alone. See U.S. Pat. No. 5,597,798 to Howell et al (1997).

Also, a variety of delivery systems have been evaluated in the context of recent chemotherapeutic therapies for patients with malignant glioma. See, for example, Charette (40), who discussed the use of biodegradable polymer wafers, chemical alteration of the blood-brain barrier, high-dose hormonal therapy, and bone marrow transplantation as new approaches to treatment. The use of growth factors and improved antiemitics also, as reported, allow patients to tolerate aggressive chemotherapy.

D. Treatment of Gliomas with Tyrosine Kinase Inhibitors

Tyrosine kinases are critical enzymes in regulating cellular growth and differentiation and are also deeply involved in oncogenesis: they are frequently activated in a variety of human cancers. Several published articles have evaluated the effects of various tyrosine kinase inhibitors on the growth and infiltrative activity of glioma cells. For example, as discussed below, it has been reported Tyrphostin AG 1478 preferentially inhibits human glioma cells expressing truncated rather than wild-type epidermal growth factor receptors (24).

The therapeutic use of tyrosine kinase inhibitors also is reviewed by Uehara (43) who described compounds that inhibit EGFR with high specificity. Their antitumor effects on EGFR-overexpressing cells and in vivo tumor models were described, including compounds that preferentially inhibit human glioma cells expressing truncated rather than wild-type EGFR. In particular, tyrphostin analogs that inhibit angiogenesis and thereby suppress tumor cell growth were discussed.

In another study (34), cocultures of human glioblastoma spheroids (derived from three separate patients) and fetal rat brain aggregates were examined for the extent of infiltration in the presence of 0 to 100 μmol/L genistein, which is a tyrosine kinase (TK) inhibitor, and 3 μmol/L tyrphostin A25, which is a specific EGFR-TK inhibitor. Infiltration (not attachment) was completely inhibited by genistein at 10 μmol/L, the IC20 for monolayer growth inhibition in two cell lines. Tyrphostin A25 at 3 μmol/L (the IC20 for monolayers) reduced invasion in a third cell line from 38.8+/−6.1% invasion-hour per hour. However, no change was detected in EGFR-associated tyrosine phosphorylation at those doses in monolayers by ³²P immunolabeling, an observation attributed to the known effects of low concentrations of TK inhibitors. These authors concluded that their data showed that invasion can be effectively inhibited at much lower concentrations of TK inhibitors than are necessary for growth suppression.

Nishiya et al. (36) reported that herbimycin A, a potent tyrosine kinase inhibitor, suppressed nitric oxide synthase (NOS) induced by lipopolysaccharide (LPS) and interferon-garnma (IFN-gamma) in C6 glial cells. These authors concluded that their results suggest that herbimycin A suppresses iNOS induction by inhibition of both NF-kappa B activation caused by LPS, and tyrosine-phosphorylation of JAK2 caused by IFN-gamma in C6 glioma cells.

Oude Weemink et al. (45) tested the effects of specific protein tyrosine kinase inhibitors on growth factor-induced DNA synthesis and on glioma cell proliferation. Whereas genistein inhibited both EGF- and platelet derived growth factor (PDGF)-stimulated autophosphorylation of the receptors and induction of DNA synthesis, it was found to be cytotoxic to the cells. The tyrphostins RG 50875 and RG 13022 were found to dose-dependently inhibit DNA synthesis induced by EGF, PDGF, and serum. RG 13022 completely blocked the EGF- and PDGF-induced DNA synthesis at a concentration of 50 μmol/L. These tyrphostins showed no selectivity in blocking either EGF or PDGF signaling. With concentrations up to mumol/L, no cytotoxic side effects of the tyrphostins were observed. Both tyrphostins also inhibit serum-driven cell growth in a dose-dependent manner.

Other tyrosine kinase inhibitors are described in U.S. Pat. No. 5,712,395 to App et al. (1998) and U.S. Pat. No. 5,710,173 to Tang et al. (1998). These include a variety of small molecules.

E. Treatment of Gliomas by Inhibiting Tyrosine Kinase-Mediated Downstream Enzymatic Activities

Relevant studies also have evaluated down-stream effects of tyrosine kinase mediated cellular signaling pathways. For example, it is known that glioma cells have very high protein kinase C (PKC) enzyme activity when compared to non-malignant glia, and that their PKC activity correlates with their proliferation rate (35). These authors treated established human glioma cell lines A172, U563 or U251 with either the highly selective PKC inhibitor CGP 41 251, or with genistein, a tyrosine kinase inhibitor. They reported that the proliferation rate and PKC activity of all the glioma lines was reduced by CGP 41 251, and that the IC50 values for inhibiting cell proliferation corresponded to the IC50 values for inhibition of PKC activity. Genistein also was reported to have inhibited cell proliferation, with IC50 proliferation values approximating those for inhibition of tyrosine kinase activity in cell free protein extracts.

Notably, in genistein-treated cells, downstream PKC enzyme activity was dose dependently reduced such that the correlation coefficient for effects of genistein on proliferation rate and PKC activity was 0.92. The authors suggested a conclusion that upstream tyrosine kinase linked events, rather than an autonomously functioning PKC, resulted in the high PKC activity observed in glioma. They also concluded that proliferative signal transduction in glioma cells occurs through a predominantly PKC-dependent pathway and that selectively targeting this enzyme provides an approach to glioma therapy.

In a related study, Pollack et al. (38) examined the activity of seven-hydroxystaurosporine (UCN-01), which is a derivative of the nonselective protein kinase inhibitor staurosporine and which exhibits significant selectivity for protein kinase C (PKC), as an inhibitor of proliferation in three established and three low-passage malignant glioma cell lines in vitro. They reported finding a striking inhibition of proliferation with a median effective concentration of 20 to 100 nM. In addition, UCN-01 enhanced the inhibition of glioma cell proliferation achieved with conventional chemotherapeutic agents, exhibiting synergistic effects with cisplatin and additive effects with 1,3-bis(2-chloroethyl)-1-nitrosourea. In vivo studies in which UCN-01 was administered by continuous intraperitoneal infusion in subcutaneous and intracranial intraparenchymal nude rat models demonstrated significant activity against U-87 glioma xenografts at dose levels that were well tolerated. The authors concluded that UCN-01 is an effective agent for the inhibition of glioma proliferation in vitro and in vivo and has potential for clinical applicability in the treatment of human gliomas.

U.S. Pat. No. 5,744,460 to Muller (1998) also describes a combination treatment for proliferative diseases involving the administration of an oligonucleotide or oligonucleotide derivative targeted to PKC, especially PKC- alpha, together with one or more other chemotherapeutic agents.

In a study by Zenke et al. (44), the Ca2+influx into the cytoplasm of arteriolar smooth muscle cells in the tumor, mediated through the tyrosine kinase pathway, was modulated by the administration of diltiazem. The intra-tumor concentration of Nimustine-HCl (ACNU) with co-administration of diltiazem was significantly increased compared to that without the co-administration. Co-administration of diltiazem also may be a valuable strategy in chemotherapy for glioma by enhancing the selective increase of intra-tumor concentration of the anti-cancer drug.

### SUMMARY OF THE INVENTION

The present inventors have discovered that the expression of mutant epidermal growth factor receptor (EGFR) genes in cancer cells can suppress the apoptosis inducing activity of chemotherapeutic agents. Thus, the present invention provides compositions and methods for the modulation of the apoptosis-inhibiting effects of such mutant EGFR gene products to enhance the efficacy of, for example, conventional anti-tumor therapies, particularly chemotherapy, that induce apoptosis in the tumor cells. Accordingly, the present invention also provides methods and compositions, particularly combination therapies and combination pharmaceutical compositions, that are useful in the treatment of disorders in which a mutant EGFR inhibits the increased rate of apoptosis induced by various therapies. The present invention is of particular relevance in the treatment of cancer, such as malignant gliomas, in which drug resistance mediated by mutant EGFR genes is common.

The use of tyrosine kinase inhibitors in the invention's methods and compositions, particularly those that are specific for ΔEGFR or some other tumor specific EGFR, are preferred. Such compositions preferably are used in combination with other therapeutic agents, particularly other chemotherapeutic agents, and with other therapies, such as radiotherapy, which induce apoptosis or increase the rate of apoptosis in the target cells in a host or patient, particularly human patients. Such other chemotherapeutic agents are described in detail below.

### BRIEF DESCRIPTION OF THE DRAWINGS

FIG. 1(A) shows quantitation of EGFR species in cells. Western blot analysis of expression (lower) and auto-phosphorylation (upper) of wt EGFR (♦) and ΔEGFR (●) in U87MG, U87MG.ΔEGFR, U87MG.DK and U87MG.wtEGFR cells grown in media containing 10% serum. Low and similar levels of endogenous wt EGFR expression was also detected in U87MG, U87MG.ΔEGFR and U87MG.DK at longer exposures (data not shown). FIG. 1(B) shows survival of U87MG (□), U87MG.ΔEGFR (⋄), U87MG.DK (∘) and U87MG.wtEGFR (Δ) cells in response to varying amounts of cisplatin (CDDP). Cells were plated in triplicate 60-mm dishes and treated with media containing various concentrations of CDDP for 1 hr, followed by incubation with fresh media for 10 to 12 days. Numbers of colonies were counted after Giemsa staining. Results were reproduced in four independent experiments; bars, SID.

FIG. 2 shows that cells expressing ΔEGFR are diminished in their apoptotic response to CDDP. Cells were seeded on cover slips, treated with 5 μg/ml of CDDP for 2 days, and then TUNEL assays were performed. The percent of TUNEL-positive cells induced by CDDP treatment was assessed by counting more than 400 cells per coverslip. □, U87MG; ▪, U87MG.ΔEGFR;_, U87MG.DK;_, U87MG.wtEGFR. Values represent means from three independent experiments each performed in triplicate; (***) significantly different (p <0.001) from the value for the U87MG.ΔEGFR cells treated with CDDP; bars, SE.

FIG. 3 shows that cells expressing ΔEGFR have higher initial and sustained levels of BclX_{L}. Western blot analysis of BclX_{L} expression in U87MG, U87MG.ΔEGFR, U87MG.DK and U87MG.wtEGFR cells was performed after CDDP treatment. Total cell lysates were prepared at the various time points indicated. For each sample, 20 μg of lysate protein was used. Results were reproduced in three independent experiments.

FIG. 4 shows that ΔEGFR expression causes reduced activation of caspase-3-like proteases in response to CDDP treatment. (A) Total cell lysates were prepared 2 days after treatment with 5 μg/ml CDDP. Samples were assayed for protease activity using the peptide substrate, Ac-DEVD-pNA. For inhibition of the protease activity, 10 μM Ac-DEVD-CHO was added to the reaction mixture prior to the addition of substrate. Cells were also treated with the plasma membrane-soluble caspase inhibitor Z-Asp-CH₂DCB (200 μM) prior to and during CDDP treatment. □, U87MG; ▪, U87MG.ΔEGFR;_, U87MG.DK;_, U87MG.wtEGFR. Values represent the means of seven independent experiments (two for Z-AspCH2-DCB experiment); (***) significantly different (p <0.001) from the value for U87MG.ΔEGFR cells treated with CDDP; bars, SE. (B) Proteolytic cleavage of PARP after CDDP treatment. For each sample, 20 μg total clarified protein lysate was loaded on to SDS-PAGE gels, electrophoresed, transferred to membranes and probed with anti-PARP monoclonal antibodies. FL, full-length; CF, cleaved fragment.

FIG. 5 shows that overexpression of BclX_{L} inhibits the caspase-3-like protease activation and induction of apoptosis caused by CDDP treatment. (A) Western blot analysis showing expression levels of BclX_{L} in U87MG Bcl-X_{L} overexpressing clones (Bcl-X_{L}-6, -9, -13, -11 -12, and -8) and empty vector-transfected clones (SFFV-2 and -5). For each sample, 20 μg of total cell lysate was used. (B) Caspase-3-like protease activity of the BclX_{L}-overexpressing clones 2 days after treatment with 5 μg/ml CDDP measured as described in FIG. 4A. Values are means of two to six independent experiments; bars, SE. (C) Apoptosis rate of Bcl-X_{L} overexpressing clones 2 days after CDDP 5 μg/ml treatment determined by TUNEL assay. Values represent means from two to three independent experiments in triplicates; bars, SE.

FIG. 6 shows that modulation of CDDP-induced apoptosis in U87MG.ΔEGFR cells by various tyrphostin-type tyrosine kinase inhibitors. (A) Reduced Bcl-X_{L} expression upon exposure to tyrphostin AG1478. U87MG.ΔEGFR cells were treated with the ΔEGFR-selective tyrphostin AG1478 (15 μM) with or without CDDP (5 μg/ml) and total cell lysates were prepared. For each sample, 20 μg of lysate was used. (B) Increased apoptosis induced by combination treatment using ΔEGFR-selective tyrphostins (AG1478 or AG1517) and CDDP. U87MG.ΔEGFR cells were treated with or without CDDP 5 μg/ml for 2 days in the presence of the ΔEGFR-selective tyrphostins (AG1478 or AG1517) or the non-specific, nonpotent tyrphostins (AG1479 or AG1536) at the concentrations indicated. For control, the vehicle (DMSO) was used. Apoptosis rate determined by TUNEL assays of triplicate cover slips. Experiments were repeated independently two to three times, with similar results.

### MODES OF CARRYING OUT THE INVENTION

The present invention is based on the discovery that ΔEGFR expression in glioma cells confers resistance to some commonly utilized chemotherapeutic agents. The observed resistance was associated with suppression of drug-induced apoptosis, which was largely mediated by increased expression of Bcl-X_{L} and subsequent inhibition of caspase-3-like protease activation. These effects required constitutive signaling by ΔEGFR, since overexpression of kinase-deficient ΔEGFR (DK) or wt EGFR had no such effects. Moreover, inhibition of ΔEGFR function by inhibitors to the ΔEGFR mutation sensitized the cells to drug treatment. See, generally, the abstract of Nagane et al., published in the Proceedings of an AACR Special Conference entitled “Molecular Mechanisms of Apoptosis Regulation” held on Jan. 9-13, 1998, the disclosure of which is hereby incorporated by reference.

These results suggest a new treatment strategy for glioma and other tumors in which the inhibitionof ΔEGFR (or other relatively tumor specific EGFR mutation) is effectively coupled with chemotherapy.

The compositions, agents and methods that are discussed and exemplified below relate to the modulation of ΔEGFR mediated intracellular pathways. However, these compositions, agents and therapeutic methods are also relevant to other tumor specific EGFR mutant genes that inhibit the induction of apoptosis or the increased rate of apoptosis associated with various chemotherapies and radiotherapy. The particular mutation of the EGFR gene is not critical to the invention, although the administration of agents that are relatively selective for tumor specific EGFR mutations are prefered because they will reduce or avoid any deleterious side effects that may result from the use of agents that also modulate the activity of wild type EGFRs. The agents, methods and compositions of the present invention are thus of particular value in the treatment of conditions in which target cells or target tissues exhibit resistance to therapies that would otherwise lead to drug-induced apoptosis, primarily where such resistance is mediated by increased expression of Bcl-X_{L} and subsequent inhibition of caspase-3-like protease activation.

As described herein, the present invention is useful in the modulation of ΔEGFR mediated inhibition of cellular apoptosis. As used herein, modulation of apoptosis means increasing or decreasing the number of cells that would otherwise undergo apoptosis in a given cell population. This can be effected by modulating (increasing or decreasing) one or more interactions of ΔEGFR with its intracellular substrates. Preferably, the given cell population in which apoptosis is to be modulated is found in a tumor or other tissue or group of cells in which beneficial effect results from the modulation. Also, preferably, the increase or decrease in number of cells that would otherwise undergo apoptosis in a given cell population is at least about 10%, 20%, 40% or more and preferably is at least about 50% of the cells in that population.

As used herein, an agent is considered to be relatively specific for a mutant EGFR when it has a relatively greater affinity for such a mutant than for a wild type EGFR. Prefered agents will be substantially more specific for mutant forms of EGFR and will be relatively specific for mutant forms of EGFR that are substantially tumor specific.

I. General Description

Alterations of the epidermal growth factor receptor (EGFR) gene occur frequently in human malignant gliomas, the most common of which is deletion of exons 2-7, resulting in truncation of the extracellular domain (ΔEGFR or EGFRvIII). This receptor has been shown to markedly enhance tumorigenicity, in part by decreasing apoptosis through up-regulation of Bcl-X_{L}. The present invention is based on the discovery that the ΔEGFR concomitantly confers resistance to the chemotherapeutic drug cisplatin (CDDP) by suppression of CDDP-induced apoptosis.

Expression of Bcl-X_{L} was elevated in U87MG.ΔEGFR cells prior to and during CDDP treatment, whereas it decreased considerably in CDDP-treated parental cells. CDDP-induced activation of caspase-3-like proteases was significantly suppressed in U87MG.ΔEGFR cells. These responses were highly specific to constitutively kinase-active ΔEGFR, since overexpression of kinase-deficient ΔEGFR (DK) or wild-type EGFR had no such effects. Correspondingly, ablation of ΔEGFR signaling with specific tyrosine kinase inhibitors reduced Bcl-X_{L} expression and potentiated CDDP-induced apoptosis in U87MG.ΔEGFR cells. Similar to the effects seen with ΔEGFR, ectopic overexpression of Bcl-X_{L} in parental U87MG cells also resulted in suppression of both caspase activation and apoptosis induced by CDDP.

These results suggest that the expression of ΔEGFR in glioma cells confers resistance to CDDP and that this effect may be mediated by increased Bcl-X_{L} expression and subsequent suppression of caspase-3-like protease activation. Such resistance may have important clinical implications for the use of CDDP in the treatment of malignant gliomas expressing ΔEGFR.

The EGFR gene is the most frequently amplified and mutated oncogene observed in highly malignant gliomas, and the data presented herein show that overexpression of a tumor-derived mutant form of EGFR, ΔEGFR, in human U87MG glioma cells results in resistance to the chemotherapeutic drug, CDDP. Drug resistance was associated with a significant reduction in the rate of apoptosis, which could be at least partially attributed to ΔEGFR-induced, elevated Bcl-X_{L} expression and reduced caspase activity. Elevated levels of Bcl-X_{L}, known to inhibit apoptosis, were observed in U87MG.ΔEGFR cells prior to treatment, but were even more manifest after CDDP treatment. Additionally, caspase activation, a core component of the apoptosis execution machinery, was inhibited post-CDDP treatment in U87MG.ΔEGFR cells, in agreement with the recent reports that BclX_{L} regulates caspase activation (19, 25).

The foregoing observations were highly specific to cells expressing constitutively active ΔEGFR, since overexpression of the kinase-defective form of ΔEGFR, DK, or wt EGFR had no such effects, and inhibition of ΔEGFR kinase activity by the tyrphostin AG1478, a tyrosine kinase inhibitor with selectivity for ΔEGFR, significantly reversed these properties. Thus, the results described below support the growing notion that certain tumor-specific genetic alterations may bestow a growth advantage to and enhance survival of tumor cells in the presence of chemotherapeutics by reducing apoptosis levels.

Previous studies have shown that the molecular and biological effects of ΔEGFR on glioma tumorigenesis differ from those of wt EGFR (15, 16). Unlike ΔEGFR, overexpression of wt EGFR was unable to confer significant growth advantages to glioma cells both in vitro and in vivo, and also did not contribute to CDDP resistance. Several lines of evidence have suggested that enhanced expression of wt EGFR may be associated with CDDP resistance in other tumor types (26, 27). For example, suppression of EGFR activity by a dominant negative EGFR construct enhanced the cytotoxic effect of CDDP in pancreatic cancer cells (27).

In contrast, activation of EGFR by EGF has been shown to enhance sensitivity of a variety of cancer cells to CDDP (28). Dixit et al. also showed that down-regulation of EGFR expression by antisense RNA resulted in CDDP resistance of breast cancer cells expressing high level of wt EGFR, but not in those expressing low levels (29). The observation that intensive stimulation of overexpressed wt EGFR in A431 cells induced apoptosis, whereas lower levels of receptor phosphorylation stimulated proliferation (30), however, implies that the activation level of overexpressed wt EGFR may be critical for its biological consequence.

Since the extent of autophosphorylation per molecule of ΔEGFR was only 10% that of wt EGFR fully activated by EGF (15), the low level of autophosphorylation of ΔEGFR and its constitutive signaling may play an important role in conferring CDDP resistance. Alternatively, ΔEGFR could transduce signals through pathways different from those of wt EGFR, since transcriptional up-regulation of Bcl-X_{L} was only observed in cells overexpressing ΔEGFR, but not in those overexpressing wt EGFR or DK (16). It is also possible that ΔEGFR may preferentially phosphorylate tyrosine residues of molecules involved in apoptosis regulation. In this regard, we have demonstrated that ΔEGFR is phosphorylated and is associated with the adaptor protein Shc (31), thus leaving open the possibility that Shc Y239 and Y240, which have been shown to mediate anti-apoptotic signals (32), are potential substrates of ΔEGFR.

The data discussed herein show that combination treatment of U87MG.ΔEGFR cells with the mutant EGFR-specific tyrosine kinase inhibitor AG1478 and CDDP synergistically induced apoptosis. Since tumor-derived ΔEGFR conferred resistance of tumor cells to drug-induced apoptosis, which was specific to ΔEGFR but not to wt EGFR, it is reasonable to consider ΔEGFR as a therapeutic target to enhance the efficacy of drug treatment.

Thus, it would be of benefit to render ΔEGFR-positive tumor cells susceptible to treatment through specific targeting and combination chemotherapy. Monoclonal antibodies raised against ΔEGFR-specific epitopes have been produced for potential use in imaging and for immunotoxin delivery (33). Tyrphostin AG1478 has also been shown to preferentially inhibit constitutive autophosphorylation of ΔEGFR and growth of tumor cells expressing ΔEGFR (24). The experimental work discussed below shows that treatment of U87MG.ΔEGFR cells with AG1478 alone resulted in the reduction of resistance-related BclA_{L} expression to basal levels, but was not enough to induce apoptosis. However, tyrphostin treatment in combination with CDDP was able to induce apoptosis synergistically and more effectively in U87MG.AEGFR cells than did CDDP treatment alone. These results provide a fundamental basis for the development of combination treatments for proliferative diseases, such as the cancers of the lung, breast and brain, and in particular intractable malignant gliomas, especially those expressing ΔEGFR.

II. Specific Embodiments

A. Combination Therapy

Agents of the present invention that modulate tyrosine kinase activity are intended to be administered in combination with another agent or agents that modulate (particularly that inhibit) unwanted cellular proliferation. For example, an agent of the present invention that inhibits tyrosine kinase activity in a glioma cell can be administered in combination with anti-cancer agents in methods to control cancer cell growth. As used herein, two agents are said to be administered in combination when the two agents are administered simultaneously or are administered independently in a fashion such that the agents will act at the same time.

The inhibition of EGFR-mediated inhibition of apoptosis can be pursued in combination with conventional chemotherapies. The routes and timing for using a chemotherapeutic agent in combination with agents that the modulate EGFR-mediated inhibition of apoptosis will depend, for example, upon the particular chemotherapeutic agent used and the tumor cell type treated. Such other chemotherapeutic agents are described in detail in U.S. Pat. No. 5,744,460 which was discussed above and is specifically incorporated by reference.

Other examples of chemotherapeutic agents that can be used in combination with agents that affect EGFR-mediate effects include, but are not limited to alkylating agents, such as cyclophosphamide (CTX; cytoxan), chlorambucil (CHL; leukeran), cisplatin (CDDP; CisP; platinol) busulfan (myleran), melphalan, carmustine (BCNU), streptozotocin, triethylenemelamine (TEM), mitomycin C, and the like alkylating agents; anti-metabolites, such as methotrexate (MTX), etoposide (VP16; vepesid) 6-mercaptopurine (6MP), 6-thiocguanine (6TG), cytarabine (Ara-C), 5-fluorouracil (5FU), dacarbazine (DTIC), and the like anti-metabolites; antibiotics, such as actinomycin D, doxorubicin (DXR; adriamycin), daunorubicin (daunomycin), bleomycin, mithramycin and the like antibiotics; alkaloids, such as vinca alkaloids such as vincristine (VCR), vinblastine, and the like; and other antitumor agents, such as taxol and taxol derivatives, the cytostatic agents glucocorticoids such as dexamethasone (DEX; decadron) and corticosteroids such as prednisone, nucleoside enzyme inhibitors such as hydroxyurea, amino acid depleting enzymes such as asparaginase, and the like diverse antitumor agents. A skilled artisan can readily recognize that there is no limit as to the structural nature of the agents of the present invention.

The use of the cytotoxic agents described above in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using the present histoculture methods.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

To the extent that the present invention further provides compositions containing one or more tyrosine kinase inhibitors or other agents that modulate the apoptosis inhibiting activities of mutant EGFR molecules. Such agents also can be administered in dosages known to be effective or that can readily be determined as effective by one skilled in the art.

In general, the agents of the present invention can be administered via parenteral, subcutaneous, intravenous, intramuscular, intraperitoneal, transdermal, or buccal routes. Alternatively, or concurrently, administration may be by the oral route. The dosage administered will be dependent upon the age, health, and weight of the recipient, kind of concurrent treatment, if any, frequency of treatment, and the nature of the effect desired. For example, to treat tumor cells, the compositions of the present invention may be administered systemically or locally to the individual being treated. As will be well known to the skilled artisan, there are many methods that can readily be adapted to administer such agents.

In addition to the pharmacologically active tyrosine kinase inhibiting agents and chemotherapeutic agents, the compositions of the present invention may contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient. Suitable formulations for oral administration include hard or soft gelatin capsules, pills, tablets, including coated tablets, elixirs, suspensions, syrups or inhalations and controlled release forms thereof.

In practicing the methods of this invention, the two-component compositions of this invention may be used together or in further combination with other therapeutic or diagnostic agents. In its preferred embodiments, the two primary components of this invention (i.e., a tyrosine kinase inhibitor and an apoptosis inducing or apoptosis rate increasing therapy) may be coadministered along with yet other compounds typically prescribed for these conditions according to generally accepted medical practice, such as various other chemotherapeutic agents.

B. General Description of Materials and Methods Used Below:

Chemicals.

cisplatin (CDDP), paclitaxel (Taxol), and vincristine were purchased from Sigma (St. Louis, Mo.). Tyrphostin AG1478 was obtained from Calbiochem (La Jolla, Calif.). Tyrphostins AG1479, AG1517, and AG1536 were chemically synthesized at The Hebrew Univ. of Jerusalem.

Cells and Culture.

The human glioma cell line U87MG which expresses low amount of wt EGFR, and its sublines, U87MG.ΔEGFR, U87MG.DK, and U87MG.wtEGFR overexpressing ΔEGFR, a kinase-deficient mutant of ΔEGFR (DK), and exogenous wt EGFR, respectively, were described previously (15). U87MG cells were transfected with either pSFFVneo-bcl-X_{L} or its control vector pSFFV-neo plasmids (gifts of Dr. S. J. Korsmeyer, Washington Univ. St. Louis, Mo.) using the calcium phosphate precipitation method and selected in the presence of 400 μg/ml G418 (Gibco/BRL, Grand Island, N.Y.). Clones expressing high levels of Bcl-X_{L} were used for experiments. All cells were cultured as described (16).

Cell Survival Assay.

To determine the level of resistance of the cells to chemotherapeutic agents, colony forming efficiency assays were performed as described (20). Briefly, 400 or 700 cells were seeded in triplicate into dishes having a 60-mm diameter. On the next day cells were treated with various concentrations of CDDP for 1 hr or Taxol or vincristine for 2 hr, and then further incubated for 10 to 12 days in the fresh medium. The cells were fixed in methanol, stained with Giemsa solution (Sigma) and the number of colonies containing greater than 50 cells were counted. The survival fraction was calculated as the ratio of the colony-forming efficiency of drug-treated and untreated cells. Drug-resistance was expressed as an IC50 value (in μM), the dose resulting in 50% survival.

In Situ (TUNEL) Labeling of Apoptotic DNA Fragmentation.

Apoptotic cells were detected by use of terminal deoxynucleotidyl transferase mediated nick end labeling (TUNEL) of apoptotic DNA strand breaks as described (16). Apoptotic index was calculated as a ratio of apoptotic cell number to total tumor cell number within the areas by counting more than 400 cells in each coverslip.

Caspase Activity Assay.

Cells were lysed in lysis buffer (ApoAlert, Clontech, Palo Alto, Calif.) for 15 min on ice, followed by centrifugation. Protease assays included 89 μl of reaction buffer (100 mM HEPES, pH 7.5, 20% v/v glycerol, 6 mM dithiothreitol, and 0.5 mM EDTA), I μl of 10 mM acetyl-Asp-Glu-Val-Asp-p-nitroanilide (Ac-DEVD-pNA) in DMSO (California Peptide Research, Inc., Napa, Calif.), and 10 μl of equal protein cell lysate. Samples were incubated at 37° C., and enzyme-catalyzed release of p-nitroanilide (pNA) was monitored at 405 nM for 1 h in a microtiter plate reader (Molecular Devices Inc., Sunnyvale, Calif.). Aliquots of cell lysates diluted into the reaction buffer were first incubated with 10 μM caspase-3-like protease inhibitor, Ac-Asp-Glu-Val-Asp-aldehyde (Ac-DEVD-CHO) (Bachem, Torrance, Calif.), at 37° C. for 30 min. Values for picomoles of pNA released were calculated from those observed in OD₄₀₅ values using a standard curve of pNA. One unit of protease activity was defined as the amount of enzyme required to release 1 pmol pNA/min at 37° C. For in vivo caspase inhibition studies, cells were cultured in the presence of peptide caspase inhibitor Z-Asp-CH2-DCB (Bachem).

Western Blotting.

Cell lysates were prepared in RIPA buffer (16), equal protein per sample was separated on SDS-polyacrylamide gels, and transferred to nitrocellulose membranes (BioRad, Hercules, Calif.). Proteins on the membranes were probed with antibodies to Bcl-X_{L} (Transduction, Lexington, Ky.), poly(ADP-ribose) polymerase (PARP) (C2-10; Enzyme System Products, Dublin, Calif.), EGFR (C13) (15), or phosphotyrosine (4G10, Upstate Biotechnology, Lake Placid, N.Y.), and detected by chemiluminescence (Amersham).

### EXAMPLES

### Example 1

### ΔEGFR Confers CDDP Resistance in U87MG Human Glioma Cells

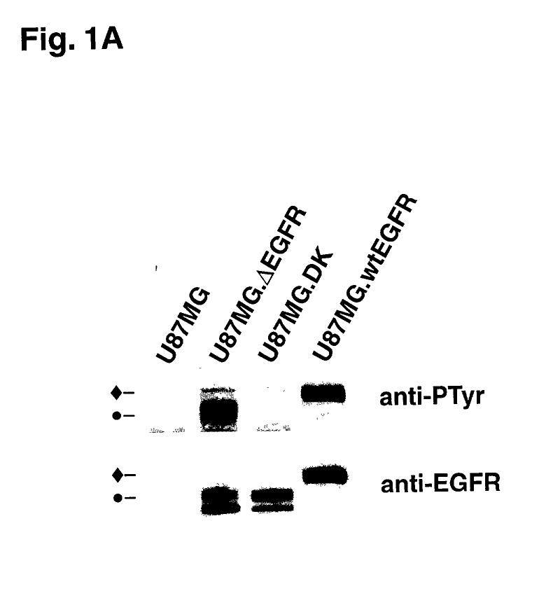

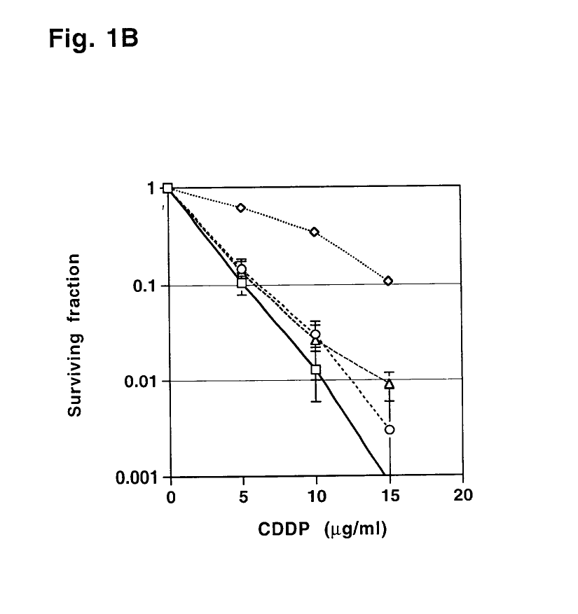

Because ΔEGFR expression in glioma cells has been shown to decrease the rate of apoptosis both under serum-starved culture conditions and in implanted tumors derived from such cells (16), the sensitivity of ΔEGFR- expressing glioma cells to the chemotherapeutic drug, CDDP, a DNA damaging agent known to induce apoptosis in tumor cells was tested (21, 22). Endogenous and exogenous wt EGFR were moderately autophosphorylated under the culture condition used (10% serum), suggesting that wt EGFR was constantly stimulated by ligand in the serum (FIG. 1A). Consistent with previous studies, ΔEGFR was also constitutively autophosphorylated while a kinase-deficient mutant of ΔEGFR (DK) was devoid of any significant tyrosine phosphorylation (15). CDDP-treated U87MG.ΔEGFR, but not U87MG.DK or U87MG.wtEGFR cells, had a significantly higher survival rate (4.3-fold greater IC50 value) than parental U87MG cells (FIG. 1B). Similar results were obtained when cells were treated with the other chemotherapeutic agents, Taxol and vincristine (data not shown). These results suggest that overexpression of active ΔEGFR confers drug resistance upon human glioma cells.

### Example 2

### U87MG.AEGFR Cells Exhibit Reduced Apoptosis upon CDDP Treatment

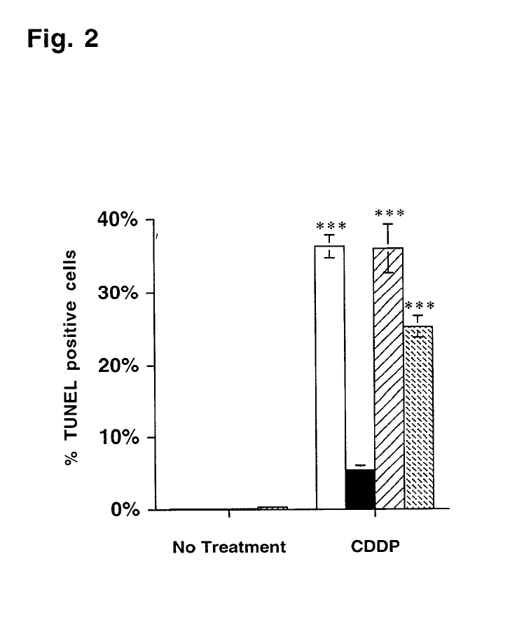

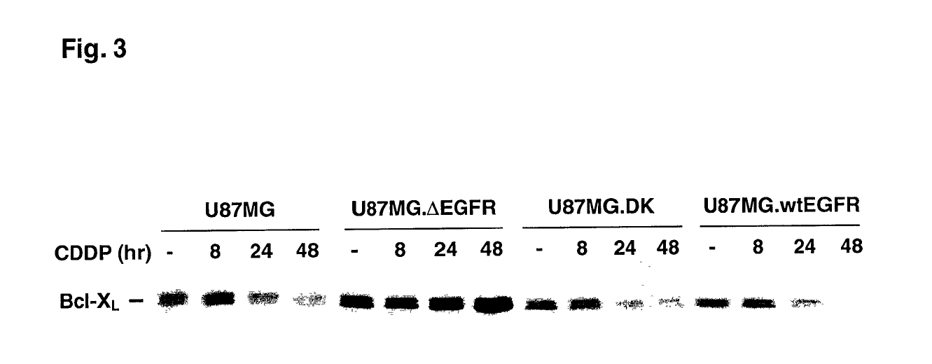

To determine whether the drug resistance observed in U87MG.ΔEGFR cells was associated with inhibition of drug-induced apoptosis, CDDP-treated glioma cells were examined for the morphologic changes typical of apoptotic cells and for their DNA fragmentation. After treatment with CDDP for 2 days, U87MG, U87MG.DK and U87MG.wtEGFR cells each displayed typical features of apoptosis such as a shrunken morphology with condensed and fragmented nuclei, whereas U87MG.ΔEGFR cells were relatively unaffected (data not shown). With regard to DNA fragmentation, the proportion of cells which were TUNEL-positive was lowest in CDDP-treated U87MG.ΔEGFR cells, being more than 5-fold lower than that of CDDP-treated U87MG, U87MG.DK, or U87MG.wtEGFR cells (all p<0.001, Student's t test) (FIG. 2). In all untreated cell lines apoptotic cells were nearly undetectable, indicating that apoptosis was induced by CDDP. Similar results were obtained in an Annexin V binding assay for apoptosis detection (data not shown). These results suggested that the constitutively active and aberrant ΔEGFR signaling may play an inhibitory role in the induction of apoptosis by the DNA damaging agent, CDDP.

### Example 3

### Increased Expression of BCI-XL, and Inhibition of Caspase Activation are Associated with Suppression of CDDP-induced Apoptosis in U87MG.ΔEGFR Cells

The mechanism by which U87MG.ΔEGFR cells were more resistant to CDDP-induced apoptosis than parental cells was investigated. Since we have previously shown the up-regulation of expression of the anti-apoptotic Bcl-X_{L} protein in U87MG.ΔEGFR cells and tumors (16), we determined whether its expression was affected by CDDP treatment. Expression levels of Bcl-X_{L} in U87MG, U87MG.DK, and U87MG.wtEGFR cells were similar and were equally decreased by continuous exposure to CDDP for more than 24 hr, whereas that in U87MG.ΔEGFR cells was higher than other cells and remained unchanged during CDDP treatment. The Bcl-X_{L} expression levels in these cells were inversely correlated with their proportion of cells undergoing apoptosis, consistent with its role as an inhibitor of cell death (FIG. 2B, 3).

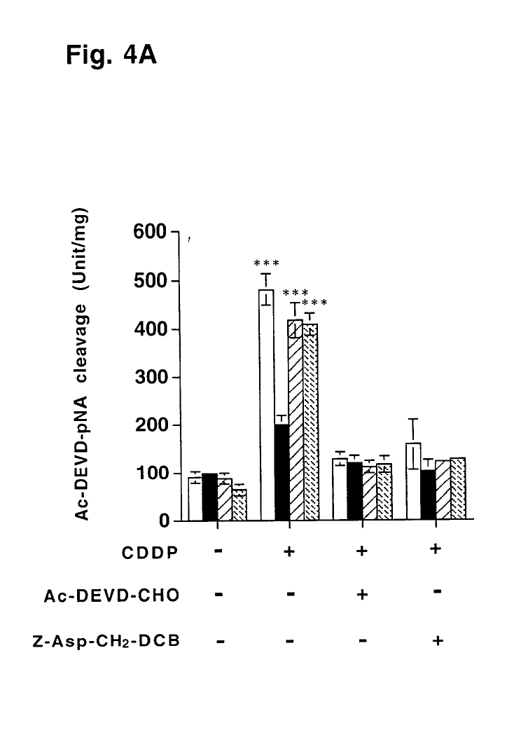

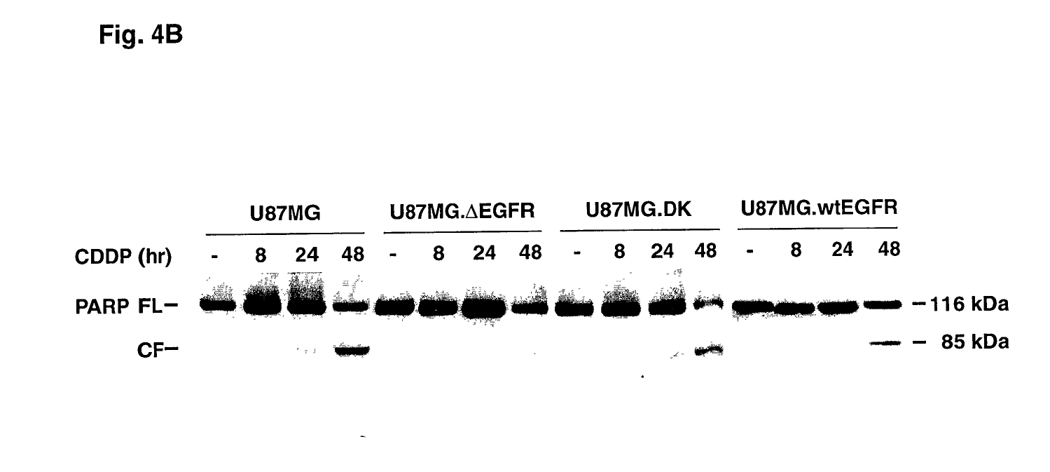

These results suggested that activation of caspases which are implicated in the execution phase of apoptosis and thought to be regulated by members of Bcl-2 family including Bcl-X_{L} (19), may be altered in U87MG.ΔEGFR cells under CDDP treatment. To test this possibility, we measured caspase-3-like protease activity using the tetrapeptide substrate, Ac-DEVD-pNA, since DEVD-specific caspase-3-like proteases play a crucial role as apoptosis executioners (19). Basal caspase-3-like protease activity in untreated cells was similar among all cell types (FIG. 4A). Forty-eight hours after CDDP treatment, a substantial increase of caspase-3-like protease activity was detected in the lysates from U87MG parental cells (5.3-fold more than basal activity). In contrast, activation of the caspase activity in CDDP-treated U87MG.ΔEGFR cells was substantially diminished (only 2.0-fold higher than its basal activity), whereas the extent of caspase-3-like protease activation in U87MG.DK and U87MG.wtEGFR cells was comparable to that in parental cells (4.7- and 6.4-fold, respectively).

The absolute caspase-3-like protease activity in U87MG.ΔEGFR cells after CDDP treatment was significantly lower (p<0.001) than that in the other cells tested. The increased caspase activities were completely abolished by pre-incubating lysates with Ac-DEVD-CHO, a specific inhibitor of caspase-3-like protease, indicating the authenticity of the measured activity (FIG. 4A). In addition, when treated with CDDP in the presence of a plasma-membrane-permeable caspase specific inhibitor, Z-Asp-CH2-DCB (23), these cells did not exhibit an increase of caspase-3-like protease activity (FIG. 4A), and also displayed a significant inhibition of CDDP-induced apoptosis (data not shown). Consistent with this result, cleavage of 116-kDa full-length PARP, a known cellular substrate of caspase-3-like proteases, into an inactive 85-kDa fragment was observed in U87MG, U87MG.DK and U87MG.wtEGFR cells at 24 hr after CDDP treatment and the degradation proceeded considerably by 48 hr, whereas in U87MG.ΔEGFR cells it only reached trace levels at 48 hr (FIG. 4B).

These results suggested that in U87MG.ΔEGFR cells, suppression of caspase-3-like protease activation was involved in the resistance to CDDP-induced apoptosis, and correlated with increased expression of BclX_{L}. Furthermore, overexpression and continuous stimulation of wt EGFR did not elicit significant effects on CDDP-induced apoptotic cell death, caspase activation, or Bcl-X_{L} expression, further suggesting that these effects were a result of expression of the tumor-derived ΔEGFR.

### Example 4

### Overexpression of Bcl-X_{L} in Parental U87MG Cells Recapitulates Suppression of both Caspase-3-like Protease Activation and Apoptosis Induced by CDDP

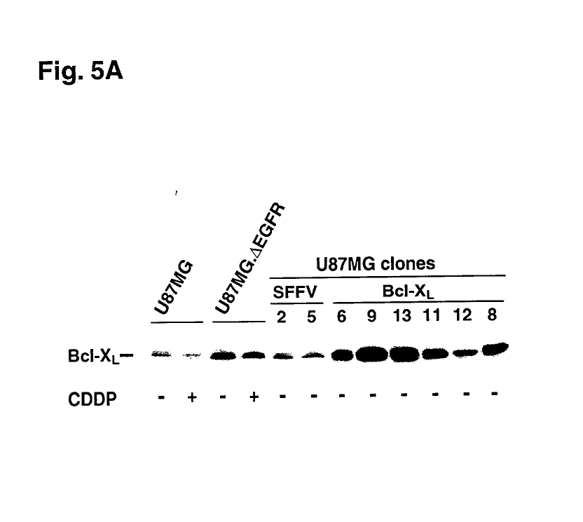

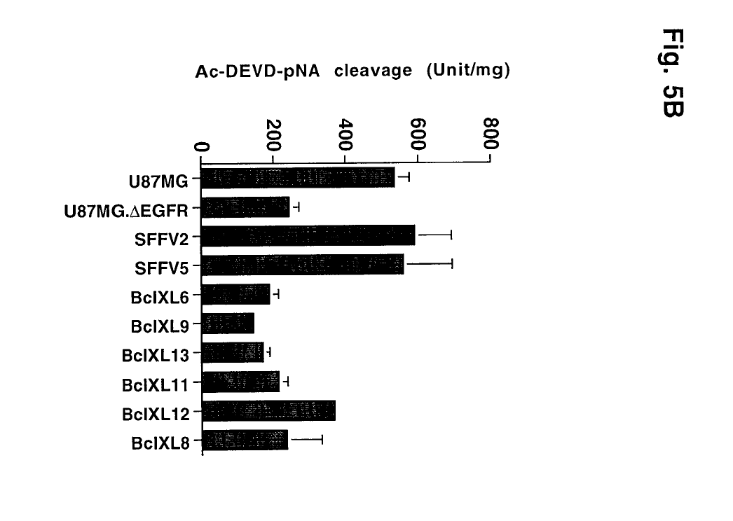

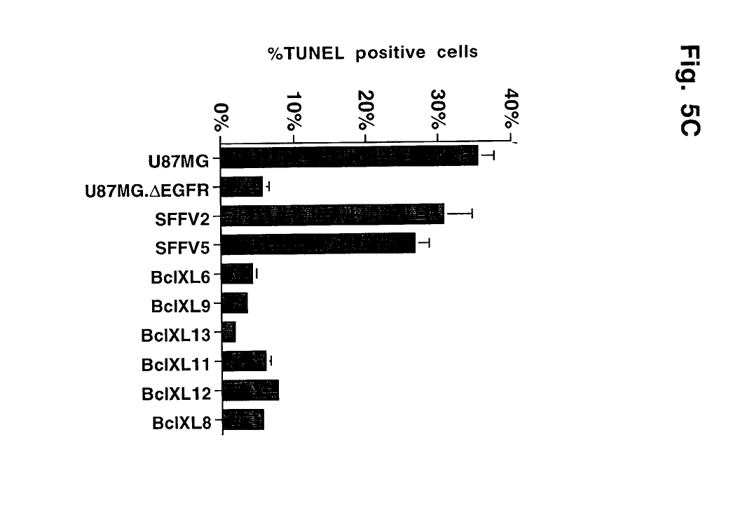

Having determined that Bcl-X_{L} expression was associated with the CDDP-resistant phenotype of U87MG.ΔEGFR cells, next tested was the question of whether Bcl-X_{L} has a direct role in CDDP-induced apoptosis. Parental U87MG cells were transfected with either the Bcl-X_{L} expression vector, pSFFVneo-bCI-X_{L}, or its control vector, pSFFV-neo, and stable clones overexpressing various levels of Bcl-X_{L} were established following G418 selection (FIG. 5A).

After CDDP treatment, U87MG.Bcl-X_{L} clones exhibited significantly lower caspase-3-like protease activities (FIG. 5B) and apoptosis indices (FIG. 5C), in an expression level-dependent manner, relative to those observed in U87MG cells (p<0.001). The caspase activity and apoptotic index in clone-12, a clone having Bcl-X_{L} overexpression at a level similar to that of U87MG.ΔEGFR cells, were also lower than in parental cells after CDDP treatment, but slightly higher than those of U87MG.ΔEGFR cells. Control vector transfectants (U87MG.SFFV) did not demonstrate inhibition of apoptosis nor caspase-3-like protease activation.

### Example 5

### The ΔEGFR-selective Tyrosine Kinase Inhibitor, AG1478, Modulates CDDP-induced Apoptosis in U87MG.ΔEGFR Cells

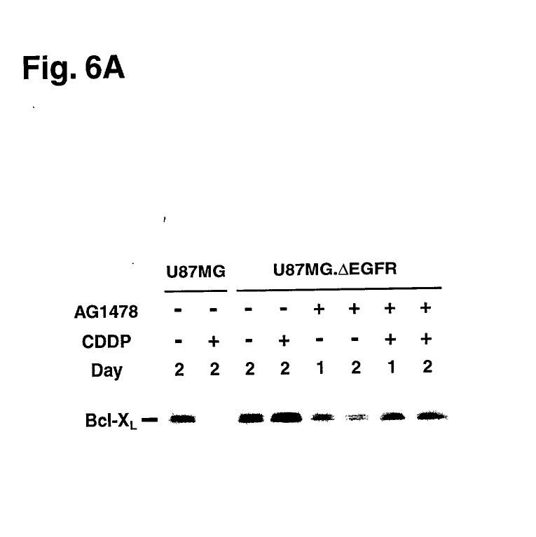

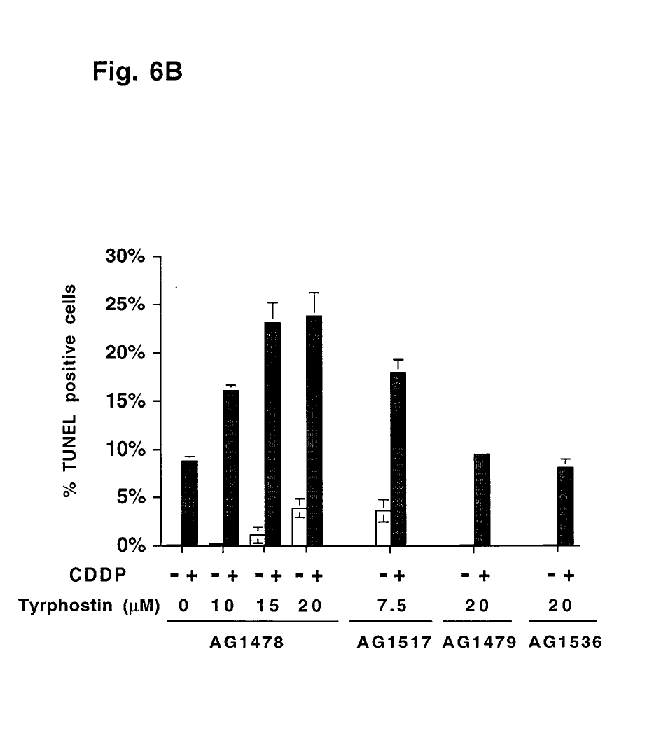

To confirm that ΔEGFR confers CDDP resistance through its signal transduction, the tyrosine kinase inhibitor AG1478 was utilized, which has been shown to have a more than 10-fold greater specificity for inhibition of ΔEGFR, relative to wt EGFR (24). Treatment of U87MG.ΔEGFR cells with 15 μM AG1478 resulted in marked reduction of Bcl-X_{L} expression (FIG. 6A). Their apoptosis index increased slightly after a single continuous treatment with 15 to 20 μM AG1478 for 2 days compared to that by the vehicle control, DMSO.

Combination treatment of U87MG.ΔEGFR with AG1478 and CDDP induced significant apoptosis in a dose-dependent and synergistic manner (p<0.001 compared with CDDP alone) (FIG. 6B). Similar results were obtained when the more potent ΔEGFR-selective tyrphostin, AG1517, was used at the lower 7.5 μM. In contrast, the non-specific and less potent tyrphostins, AG1479 and AG1536, had no effect on apoptosis induction in these cells (FIG. 6B), indicating that the observed synergistic effects were likely specific to the kinase activity of ΔEGFR. CDDP-induced caspase activation in U87MG.ΔEGFR cells was also enhanced by co-treatment with AG1478 or AG1517 (data not shown). These results suggested that constitutively active ΔEGFR signaling is responsible to Bcl-X_{L} up-regulation and inhibition of drug-induced apoptosis.

Although the present invention has been described in detail with reference to examples above, it is understood that various modifications can be made without departing from the spirit of the invention. For example, although particular tyrosine kinase inhibitors are identified above that are relatively selective for mutant EGFR, particularly for ΔEGFR, one skilled in the art will recognize that derivatives of such tyrosine kinase inhibitors are contemplated, particularly those having decreased toxicity, greater selectivity, greater bioavailability, etc. Accordingly, the invention is limited only by the following claims.

### REFERENCES

These and all other articles, publications, patents and other documents referred to throughout this application are hereby incorporated by reference in their entirety.

1. Fine, H. A. (1994) J. Neurooncol. 20, 111-120.

2. Feun, L. G., Savaraj, N. & Landy, H. J. (1994) J. Neurooncol. 20, 165-176.

3. Guchelaar, H. J., Vermes, A., Vermes, 1. & Haanen, C. (1997) Pharm. World Sci. 19, 119-125.

4. Lowe, S. W., Bodis, S., McClatchey, A., Remington, L., Ruley, H. E., Fisher, D. E., Housman, D. E. & Jacks, T. (1994) Science 266, 807-810.

5. Henriksen, R., Wilander, E. & Oberg, K. (1995) Br. J. Cancer 72, 1324-1329.

6. Nagane, M., Huang, H. J. & Cavenee, W. K. (1997) Curr. Opin. Oncol. 9, 215-222.

7. Furnari, F. B., Lin, H., Huang, H.-J. S. & Cavenee, W. K. (1997) Proc. Natl. Acad. Sci. USA 94, 12479-12484.

8. Libermann, T. A., Nusbaum, H. R., Razon, N., Kris, R., Lax, I., Soreq, H., Whittle, N., Waterfield, M. D., Ullrich, A. & Schlessinger, J. (1985) Nature 313, 144-147.

9. Schlegel, J., Merdes, A., Stumm, G., Albert, F. K., Forsting, M., Hynes, N. & Kiessling, M. (1994) Int. I Cancer 56, 72-77.

10. Ekstrand, A. J., Sugawa, N., James, C. D. & Collins, V. P. (1992) Proc. Natl. Acad. Sci. USA 9, 4309-4313.

11. Malden, L. T., Novak, U., Kaye, A. H. & Burgess, A. W. (1988) Cancer Res. 48, 2711-2714.

12. Moscatello, D. K., Holgado, M. M., Godwin, A. K., Ramirez, G., Gunn, G., Zoltick, P. W., Blegel, J. A., Hayes, R. L. & Wong, A. J. (1995) Cancer Res. 55, 5536-5539.

13. Garcia de Palazzo, 1. E., Adams, G. P., Sundareshan, P., Wong, A. J., Testa, J. R., Bigner, D. D. & Weiner, L. M. (1993) Cancer Res. 53, 3217-3220.

14. Nishikawa, R., Ji, X. D., Harmon, R. C., Lazar, C. S., Gill, G. N., Cavenee, W. K. & Huang, H. J. (1994) Proc. Natl. Acad. Sci. USA 91, 7727-773 1.

15. Huang, HA. S., Nagane, M., Klingbeil, C. K., Lin, H., Nishikawa, R., Ji, X.-D., Huang, C.-M., Gill, G. N., Wiley, H. S. & Cavenee, W. K. (1997) J. Biol. Chem. 272, 2927-2935.

16. Nagane, M., Coufal, F., Lin, H., Bögler, 0., Cavenee, W. K. & Huang, H. J. S. (1996) Cancer Res. 56, 5079-5086.

17. Ibrado, A. M., Huang, Y., Fang, G. & Bhalla, K. (1996) Cell Growth Differ. 7, 1087-1094.

18. Minn, A. J., Rudin, C. M., Boise, L. H. & Thompson, C. B. (1995) Blood 86, 1903-1910.

19. Salvesen, G. S. & Dixit, V. M. (1997) Cell 91, 443-446.

20. Nagane, M., Asai, A., Shibui, S., Nomura, K. & Kuchino, Y. (1997) Neurosurgery 41, 434-441.

21. Chu, G. (1994) J. Biol. Chem. 269, 787-790.

22. Kondo, S., Barna, B. P., Morimura, T., Takeuchi, J., Yuan, J., Akbasak, A. & Bamett, G. H. (1995) Cancer Res. 55, 6166-617 1.

23. Mashima, T., Naito, M., Kataoka, S., Kawai, H. & Tsuruo, T. (1995) Biochem. Biophys. Res. Commun. 209, 907-915.

24. Han, Y., Caday, C. G., Nanda, A., Cavenee, W. K. & Huang, H. J. (1996) Cancer Res. 56, 3859-3861.

25. Clem, R. J., Cheng, E. H.-Y., Karp, C. L., Kirsch, D. G., Ueno, K., Takahashi, A., Kastan, M. B., Gniffin, D. E., Earnshaw, W. C., Veliuona, M. A. & Hardwick, J. M. (1998) Proc. Natl. Acad. Sci. USA 95, 554-559.

26. Aboud, P. E., Hurwitz, E., Pirak, M. E., Bellot, F., Schlessinger, J. & Sela, M (1988) J. Natl. Cancer Inst. 80, 1605-1611.

27. Wagner, M., Cao, T., Lopez, M. E., Hope, C., van, N. K., Kobrin, M. S., Fan, H. U., Buchler, M. W. & Korc, M. (1996) Int. J. Cancer 68, 782-787.

28. Kroning, R., Jones, J. A., Hom, D. K., Chuang, C. C., Sanga, R., Los, G., Howell, S. B. & Christen, R. D. (1995) Br. J. Cancer 72, 615-619.

29. Dixit, M., Yang, J. L., Poirier, M. C., Price, J. O., Andrews, P. A. & Arteaga, C. L. (1997) Natl. Cancer Inst. 89, 365-373.

30. Gulli, L. F., Palmer, K. C., Chen, Y. Q. & Reddy, K. B. (1996) Cell Growth Differ. 7, 173-178.

31. Prigent, S. A., Nagane, M., Lin, H., Huvar, I., Boss, G. R., Feramisco, J. R., Cavenee, W. K. & Huang, H. J. S. (1996) J. Biol. Chem. 271, 25639-25645.

32. Gotoh, N., Tojo, A. & Shibuya, M. (1996) Embo J 15, 6197-6204.

33. Hills, D., Rowlinson, B. G. & Gullick, W. J. (1995) Int. J. Cancer 63, 537-543.

34. Penar, P., Khoshyomn S., Bhushan, A. & Tritton T. R. (1997) Neurosurgery 40(1),141-151.

35. Baltuch, G. H. & Yong, V. W. (1996) Brain. Res. 710(1-2), 143-149.

36. Nishiya, T., Uehara, T. & Nomura, Y. (1996) FEBS Lett. 371(3):333-336.

37. Chang, S. M., Barker 2nd, F. G., Huhn, S. L. Nicholas, M. K., Page, M. Rabbitt, J. & Prados, M. D. (1998) J. Neurooncol. 37(2):169-176.

38. Pollack, I. F., Kawecki, S. & Lazo, J. S. (1996) J. Neurosurg. 84(6),1024-1032.

39. Chang, S. M. & Prados, M. D. (1995) Curr. Opin. Oncol. 7(3),207-213.

40. Charette, J. L. (1995) Crit. Care. Nurs. Clin. North Am. 7(1), 135-142.

41. Balmaceda, C., Fetell, M. R. & Hesdorffer, C. (1997) Cancer Chemother. Pharmacol., 40(1),72-74.

42. Black, P., Hand, C. M., Vender, J. R. & Finkelstein, S. D. (1998) J. Neurooncol. 36(1), 7-19.

43. Uehara, Y. (1997) Gan To Kagaku Ryoho 24(11), 1536-40.

44. Zenke, K., Nakagawa, K., Kumon, Y. Ohta, S., Hatakeyama, T. & Sakaki, S. (1996) J. Neurooncol., 30(1),25-36.

45. Oude Weernink, P. A., Verheul, E., Kerkhof, E., van Veelen C. W. & Rijksen, G. (1996) Neurosurgery, 38(1), 108-13.

1. A method of modulating the apoptosis-inhibiting effect in a target cell or tissue of a mutant EGFR gene, comprising the step of administering to the cell or tissue an effective amount of a tyrosine kinase inhibitor in combination with a therapy that is effective to induce apoptosis or to increase the rate of apoptosis in the cell or tissue.

2. The method of claim 1, wherein the mutant EGFR gene is constitutively active.

3. The method of claim 2, wherein the mutant EGFR gene is ΔEGFR.

4. The method of any of claims 1 to 3, wherein the cell or tissue is a tumor selected from the group consisting of glioma, breast cancer, lung cancer and ovarian cancer.

5. The method of claim 4, wherein the tumor is a glioma.

6. The method of claim 1, wherein the apoptosis inducing or apoptosis rate increasing therapy is the administration of an agent selected from the group consisting of cisplatin, paclitaxel and vincristine.

7. The method of claim 1, wherein the tyrosine kinase inhibitor is relatively selective for a tumor specific mutant EGFR.

8. The method of claim 1, wherein the tyrosine kinase inhibitor is selected from the group consisting of Tyrphostin AG1478 and its derivatives.

9. A pharmaceutical composition comprising:
(A) an amount of an agent that is effective to induce apopotosis or increase the rate of apoptosis in a target cell or tissue; and
(B) an amount of a tyrosine kinase inhibitor that is effective to reduce the resistance to the induction of apoptosis or resistance to the increased rate of apoptosis in the target cell or tissue, the resistance being mediated by a mutant EGFR.

10. The composition of claim 9, wherein the apoptosis inducing or apopoptosis rate increasing agent is an antitumor agent selected from the group consisting of cisplatin, paclitaxel and vincristine.

11. The composition of claim 9, wherein the tyrosine kinase inhibitor is relatively selective for a tumor specific EGFR.

12. The composition of claim 9, wherein the tyrosine kinase inhibitor is selected from the group consisting of Tyrphostin AG1478 and its derivatives.

13. A kit for treating cancer comprising
(A) an amount of an agent that is effective to induce apopotosis or increase the rate of apoptosis in a target cell or tissue; and
(B) an amount of a tyrosine kinase inhibitor that is effective to reduce the resistance to the induction of apoptosis or resistance to the increased rate of apoptosis in the target cell or tissue, the resistance being mediated by a mutant EGFR.

14. The kit of claim 13, wherein the apoptosis inducing or apoptosis rate increasing agent is an antitumor agent selected from the group consisting of cisplatin, paclitaxel and vincristine.

15. The kit of claim 13, wherein the tyrosine kinase inhibitor is relatively selective for a tumor specific EGFR.

16. The kit of claim 13, wherein the tyrosine kinase inhibitor is selected from the group consisting of Tyrphostin AG1478 and its derivatives.
